(19) United States
(12) Patent Application Publication
(10) Pub. No.: US 2020/0138356 A1
(43) Pub. Date: May 7, 2020

(54) EMOTIONAL STATE MONITORING AND MODIFICATION SYSTEM

(71) Applicant: MOODIFY LTD., Kfar- Saba (IL)

(72) Inventors: Yigal SHARON, Kfar -Saba (IL); Yaniv MAMA, Kfar -Saba (IL)

(73) Assignee: MOODIFY LTD., Kfar- Saba (IL)

(21) Appl. No.: 16/177,815

(22) Filed: Nov. 1, 2018

(51) Int. Cl. A61B 5/18; A61B 5/16

(52) U.S. Cl. CPC A61B 5/18; A61B 5/165; A61B 5/0476; A61B 5/163; A61B 5/168

(57) ABSTRACT

A system for monitoring and modifying an emotional state of a subject includes at least one sensor to sense data indicative of a plurality biometric features of a subject that are related to a current emotional state of the subject. At least one stimulation device is configured to generate a stimulus to modify the current emotional state. A processor is configured to receive the sensed data, to use the sensed data to calculate at least one metric that is indicative of the current emotional state of the subject, to identify a value of the at least one metric that is indicative of a predetermined target emotional state, and, when the indicated current emotional state is different from the target emotional state, operate the stimulation device to generate a stimulus that has been previously identified as facilitating changing the current emotional state to the target emotional state.

### CROSS REFERENCE TO RELATED APPLICATIONS

The present invention claims the benefit of U.S. Provisional Patent Application No. 62/672,202, filed on May 16, 2018, which is incorporated in its entirety herein by reference.

### FIELD OF THE INVENTION

The present invention relates to a system for monitoring and modifying an emotional state of a subject.

### BACKGROUND OF THE INVENTION

Many automobile accidents are determined to have been caused by driver error. Often, in such cases, the driver is distracted or preoccupied so as not to react appropriately and safely to changing or unexpected road or traffic conditions. Emotional conditions, such as stress, anxiety, anger, boredom, and frustration, may adversely affect driving and vehicle safety. These emotional conditions may affect both drivers of private vehicles and professional drivers of commercial vehicles.

Some studies have concluded that up to 10% of some populations suffer from driving anxiety. Bad or distractive driving habits, such as texting and speaking on a mobile phone while driving, can result in accidents that may result in loss of human life or injuries. In response, various vehicle manufacturers have been incorporating technologies that warn a driver of a potentially dangerous situation, such as proximity of another vehicle or deviation from a traffic lane. In some cases, an accident prevention system may automatically control the vehicle to prevent dangerous situations or may correct a dangerous situation.

The factors that may lead to vehicle accidents may lead to accidents in other circumstances at home or in the workplace. For example, inattention during work with machinery, electricity or electrically powered devices, radiation beams, sources of heat, at heights above ground or floor level, or under other circumstances, may lead to potentially injurious or fatal accidents.

### SUMMARY OF THE INVENTION

There is thus provided, in accordance with an embodiment of the present invention, a system for monitoring and modifying an emotional state of a subject, the system including: at least one sensor to sense data indicative of a plurality biometric features of a subject that are related to a current emotional state of the subject; at least one stimulation device that is configured to generate a stimulus to modify the current emotional state; and a processor configured to receive the sensed data, to use the sensed data to calculate at least one metric that is indicative of the current emotional state of the subject, to identify a value of the at least one metric that is indicative of a predetermined target emotional state, and, when the indicated current emotional state is different from the target emotional state, to operate the at least one stimulation device to generate a stimulus that has been previously identified as facilitating changing the current emotional state to the target emotional state.

Furthermore, in accordance with an embodiment of the present invention, the at least one sensor includes an optical imaging device, and the sensed data includes an image.

Furthermore, in accordance with an embodiment of the present invention, the biometric feature includes a facial expression, and the processor is configured to analyze one or more images acquired by the optical imaging device to detect the facial expression and to calculate the at least one metric on the basis of the detected facial expression.

Furthermore, in accordance with an embodiment of the present invention, the at least one sensor includes a physiological sensor configured to sense at least one physiological parameter of the subject.

Furthermore, in accordance with an embodiment of the present invention, the physiological sensor includes a heartbeat sensor, and the physiological function includes a heartbeat or pulse of the subject.

Furthermore, in accordance with an embodiment of the present invention, the physiological sensor includes a galvanic skin response sensor, and the physiological function includes a conductivity of skin of the subject.

Furthermore, in accordance with an embodiment of the present invention, the at least one stimulation device includes a light source.

Furthermore, in accordance with an embodiment of the present invention, the processor is configured to control brightness or color of light that is emitted by the light source.

Furthermore, in accordance with an embodiment of the present invention, the at least one stimulation device includes an olfactory stimulation device configured to emit an olfactory stimulus.

Furthermore, in accordance with an embodiment of the present invention, the at least one stimulation device includes an audio generator configured to generate an audible stimulus.

Furthermore, in accordance with an embodiment of the present invention, the at least one stimulation device includes a haptic device configured to generate a tactile stimulus.

Furthermore, in accordance with an embodiment of the present invention, the at least one stimulation device includes a temperature control device.

Furthermore, in accordance with an embodiment of the present invention, the system is installable in a vehicle, wherein the subject is a driver of the vehicle.

Furthermore, in accordance with an embodiment of the present invention, the processor is further configured to use the sensed data to predict a future emotional state of the subject.

Furthermore, in accordance with an embodiment of the present invention, when the predicted indicated emotional state is a predetermined undesirable emotional state, the processor is further configured to operate the at least one stimulation device to generate a stimulus that has been previously identified as inhibiting a change of the current emotional state to the undesirable emotional state.

There is further provided, in accordance with an embodiment of the present invention, a method for monitoring and modifying an emotional state of a subject, the method including, by a processor: receiving sensed data from at least one sensor, the sensed data indicative of a plurality of biometric features of a subject that are related to a current emotional state of the subject; calculating, using the sensed data, at least one metric that is indicative of the current emotional state; identifying a value of the at least one metric that is indicative of a predetermined target emotional state; and, when the indicated current emotional state is different from the target emotional state, operating at least one stimulation device to generate a stimulus that has been previously identified as facilitating changing the current emotional state to the target emotional state.

Furthermore, in accordance with an embodiment of the present invention, a biometric feature of the plurality of biometric features includes a facial expression, and the sensed data includes one or more images acquired by an optical imaging device.

Furthermore, in accordance with an embodiment of the present invention, a biometric feature of the plurality of biometric features includes a heartbeat or skin conductivity.

Furthermore, in accordance with an embodiment of the present invention, the at least one stimulation device is selected from a group of stimulation devices consisting of: a light source, an olfactory stimulation device, audio generator, a haptic device and a temperature control device.

Furthermore, in accordance with an embodiment of the present invention, the method includes predicting a future emotional state of the subject and, when the predicted indicated emotional state is a predetermined undesirable emotional state, operating the at least one stimulation device to generate a stimulus that has been previously identified as inhibiting a change of the current emotional state to the undesirable emotional state.

### BRIEF DESCRIPTION OF THE DRAWINGS

In order for the present invention, to be better understood and for its practical applications to be appreciated, the following Figures are provided and referenced hereafter. It should be noted that the Figures are given as examples only and in no way limit the scope of the invention. Like components are denoted by like reference numerals.

FIG. 1 schematically illustrates a system for emotional state detection and modification, in accordance with an embodiment of the present invention.

FIG. 2 is a flowchart depicting a method of operation of system for emotional state detection and modification, in accordance with an embodiment of the present invention.

### DETAILED DESCRIPTION OF THE INVENTION

In the following detailed description, numerous specific details are set forth in order to provide a thorough understanding of the invention. However, it will be understood by those of ordinary skill in the art that the invention may be practiced without these specific details. In other instances, well-known methods, procedures, components, modules, units and/or circuits have not been described in detail so as not to obscure the invention.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium (e.g., a memory) that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. Unless explicitly stated, the method embodiments described herein are not constrained to a particular order or sequence. Additionally, some of the described method embodiments or elements thereof can occur or be performed simultaneously, at the same point in time, or concurrently. Unless otherwise indicated, the conjunction “or” as used herein is to be understood as inclusive (any or all of the stated options).

In accordance with an embodiment of the present invention, an emotional state modification system includes one or more sensors for sensing various biometric features or functions of a person (referred to herein as a subject) that are related to, e.g., may be affected by, an emotional state of the subject. The sensors may include one or more heartbeat or pulse sensors. Additional sensors for measuring other physiological functions, for example, breathing, perspiration or skin conductivity, body or skin temperature, muscular activity, brain activity, eye movements or blinking, or other physiological functions. In some cases, a physiological function sensor may be configured to be in physical contact with the subject in order to correctly sense the physiological function. In other cases, a physiological function sensor may be configured to remotely sense (e.g., optically, acoustically, or otherwise) the physiological function.

The sensors may include one or more optical imaging devices to record images of the subject's face or another part of the subject's body.

In some cases, sensors may measure one or more environmental factors (e.g., meteorological data, state of a vehicle, or other factors).

The system includes one or more processors that are configured to interpret biometric features of the physiological and facial image data to provide one or more emotional state metric or metric values that are indicative of a current emotional state of the subject. A processor may be configured to utilize one or more techniques for extracting an emotional state from the sensor data.

As used herein, an emotional state metric refers to any set of one or more numerical values that have been correlated with an emotional state of a subject. Typically, a method of calculation of an emotional state metric is based on analysis a large (e.g., statistically significant) sample of subjects. The analysis may include correlating objective measurements by one or more sensors (e.g., possibly including image analysis of images that are acquired by an imaging device such as a camera) with an emotional state of each of the subjects of the sample that is evaluated (possibly subjectively or using a standardized emotional state scale) concurrently with the objective measurements. The evaluation of the emotional state may include answering questions or performing tasks that are considered to be indicative of an emotional state. The evaluation may include observation by, or interaction with, an investigator with appropriate training (e.g., psychologist, psychiatrist, counselor, or similar profession or training).

For example, the processor may be configured to utilize Affectiva™ technology to recognize facial expressions of the subject in acquired facial images. The processor may relate the recognized facial expressions (e.g., on the basis of Affectiva's database relating facial expressions to emotional states) to one or more emotional states of the subject.

As another example, a processor may incorporate a Synsis™ emotion measurement engine to relate one or more physiological measurements (in some cases, one or more environmental measurements, in addition) to one or more emotional states.

In some cases, the emotional state metrics may indicate the emotional state as a multidimensional (e.g., vector) value, where each dimensional axis represents a range of emotional states between two opposite emotional states. For example, an axis may represent emotional valence (e.g., positive or pleasant emotional states versus negative or unpleasant emotional states), arousal (e.g., excited or interested versus unexcited or disinterested), dominance (e.g., controlling versus submission), engagement (e.g., expressive versus unexpressive), or other ranges of emotional states.

For example, a processor may be configured to evaluate the emotional state in accordance one or more predetermined metric scales. One or more models, e.g., based on artificial intelligence analysis of databases or otherwise, may be utilized to quantize the emotional state.

In another example, raw data (such as facial measurement) may be directly evaluated to yield an emotional state (e.g., without an intermediate in terms of other metrics).

In some cases, e.g., where is system is configured to operate with different subjects, a processor may be configured to utilize the sensor data to identify an individual subject. For example, the processor may be configured to utilize face identification technology to recognize the face of each subject (e.g., based on sensing of the face during a registration or initialization procedure). In other examples, a subject may be requested to undergo a biometric identification procedure (e.g., fingerprint or retinal scan, or other biometric identification procedure) when beginning to use the system (e.g., sit in a driver's seat or begin to operate machinery, or otherwise begin to use the system), or may be requested to enter a user identification.

With some examples of emotional state evaluation technologies, the models of the technology are previously developed using measurements on a large and varied population. Thus, the technology may be configured to identify an emotional state in most subjects without any need to train the system to recognize emotional states in each individual subject. In other examples, a subject who is using the system for the first time, or to whom the system is being adapted, may be required to participate in a training or calibration session (e.g., during which various emotional states are intentionally induced). The calibration may serve to personalize the system and accurately define a baseline emotional state and criteria for identifying deviations from the baseline state (e.g., stressful situations or other changes in emotional state).

Examples of emotional states that may be identified and quantified by an emotional state metric may include general states such as stress/anxiety, excitement, depression/low motivation, and relaxation. Quantifiable emotional states may include specific emotions such as anger, fear, sadness, joy, surprise, contempt, and disgust, and expressions such as attention, engagement, eye closure, and drowsiness.

The system includes one or more stimulation devices that are each configured to produce one or more stimuli that may affect the emotional state of the subject. Such stimulation devices may be configured to generate, for example, one or more of haptic or tactile stimulation, olfactory stimulation, visual stimulation, auditory stimulation, thermal stimulation, or another type of stimulation.

A processor of the system may be configured to operate one or more stimulation devices in accordance with an evaluated current emotional state. The processor may be configured to compare the current emotional state (e.g., as characterized by a set of one or more metrics) with a predetermined target emotional state (e.g., as defined by predetermined values of the metrics). For example, a processor may be programmed to operate a stimulation device to generate a stimulation so as to affect an emotional state of the subject. The programming may be designed to generate a stimulation that is expected to change the current emotional state of the subject toward the target emotional state.

For example, in the case of a subject who is driving a vehicle or operating machinery, the sensors of the system may detect physiological or facial features that are indicative of an emotional state (e.g., anger, boredom, aggressiveness, inattention, or other emotional state) that may be associated with unsafe driving or machine operation. In this case, the system may operate one or more of the stimulation devices to stimulate the subject in a manner that may change the subject's emotional state from a current state toward a target state. For example, a target state may be selected such that, when the current emotional state of the subject (as indicated by the emotional state metrics) is within a predetermined range of metrics that characterize the target emotional state, emotions that are associated with unsafe behavior may be reduced or eliminated.

During generation of the stimulation, the sensors may continue to operate to monitor the emotional state of the subject. The processor may be configured to control the stimulation devices based on the monitored emotional state.

For example, if the monitoring indicates that, during the stimulation, the emotional state is changing toward a target state, generation of the stimulation may continue. On the other hand, if the monitored emotional state remains unchanged or changes away from the target state (e.g., becomes more angry or inattentive, or otherwise), a different stimulus may be generated either in parallel to or instead of the previously generated stimulus.

A processor of the system may be configured with machine learning capability. The processor may thus be configured to learn the monitored effect of each generated stimulus on a particular subject. Based on the learned effects, the processor may reconfigure itself to generate those stimulations that had been previously most effective in changing that subject's emotional state toward the target state.

When the monitoring indicates that the current emotional state is similar or identical to the target state, in some cases the processor may be configured to continue the stimulation (e.g., at a reduced intensity or without change), e.g., to assist in maintaining the target state. In other cases, the processor may be configured to stop generation of the stimulation after the target emotional state is attained. For example, the processor may be configured to continue or stop generation of the stimulation based on an intrusiveness of the stimulation (e.g., how disruptive the stimulation is to the subject or to others in the vicinity of the subject), to a cost of the stimulation (e.g., consumption of a limited resource, such as a liquid scent or electrical power from a battery, or other cost), likelihood of the subject becoming desensitized (temporarily or permanently) to the stimulation, or on other considerations.

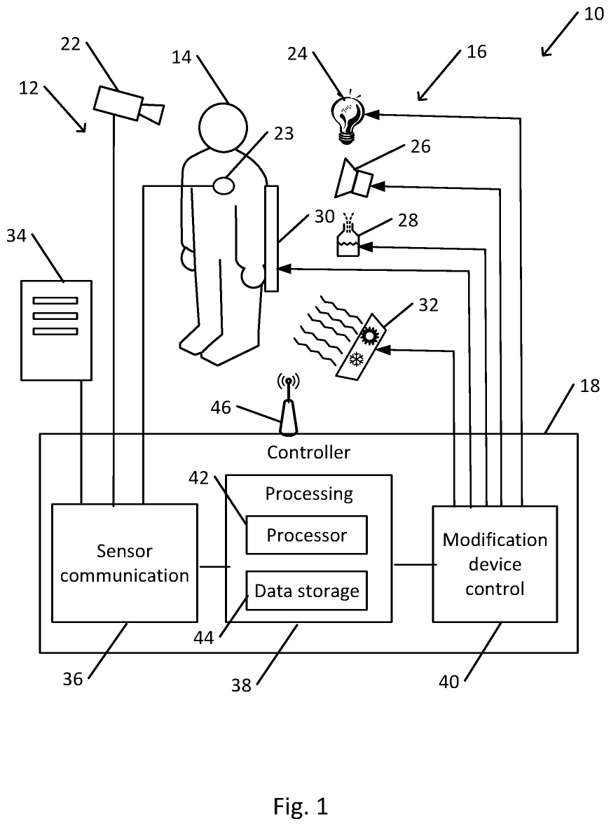

FIG. 1 schematically illustrates a system for emotional state detection and modification, in accordance with an embodiment of the present invention.

Emotional state system 10 may include one or more subject state sensors 12 that are configured to monitor one or more measurable factors that are indicative of an emotional state of a subject 14. A system controller 18 of emotional state system 10 may be configured to evaluate measurements by subject state sensor 12 to calculate one or more emotional state metrics. System controller 18 may be configured to compare the calculated emotional state metrics with a predetermined target emotional state. For example, the target emotional state may represent a range of emotional state metric values that have been predetermined as being desirable for subject 14 when engaged in a current activity (e.g., operating a vehicle or machinery, engaged in a task requiring alertness, concentration, or composure, or another type of activity). If the calculated emotional state metrics are different from the target emotional state, system controller 18 may operator one or more modification stimulation devices 16 in a manner that is configured to modify the emotional state of subject 14 to greater conformity with the target emotional state.

Emotional state system 10 may be installed in a vehicle, in the vicinity of machinery, in a factory, home, or office, or in another environment in which a subject 14 may be present. In particular, emotional state system 10 may be installed in an environment where subject 14 is expected to engage in activities where the performance of subject 14 may be affected by an emotional state of subject 14.

In some cases, emotional state system 10 may be configured to utilize one or more devices that are found in the environment of subject 14. For example, where a processor or controller is configured to operate one or more devices or components prior to installation of emotional state system 10 (e.g., a temperature control or air conditioning system, an audio or entertainment system, or another device, component, or system), emotional state system 10 may be configured to utilize or communicate with those devices or components. In some cases, all components of emotional state system 10 may be dedicated components that are configured for use as part of emotional state system 10.

Emotional state system 10 includes one or more subject state sensors 12. Subject state sensors 12 are configured to sense one or more facial features or physiological parameters of subject 14. Subject state sensors 12 may communicate with system controller 18 of emotional state system 10. For example, a subject state sensor 12 may communicate with system controller 18 via a wired (e.g., electrical cable or fiber optic) communications channel, or via a wireless communications channel.

For example, subject state sensors 12 may include one or more optical imaging devices 22. An optical imaging device 22 may include one or more cameras, optical scanners, or other imaging devices, each configured to acquire images in one or more spectral ranges. In some cases, an optical imaging device 22 may include an aiming or tracking mechanism configured to enable continuous imaging of a part of subject 14, e.g., a face of subject 14. In some cases, optical imaging device 22 may include a preexisting (e.g., before installation of emotional state system 10) security or monitoring camera configured to acquire images of a work area, operator compartment or cabin, a driver or passenger cabin or compartment, or of another are where subject 14 may be present.

For example, an optical imaging device 22 may include a camera that operates in the visible spectral range and that is configured to continuously acquire images (e.g., as a sequence of still images or of video frames) of subject 14. The camera may be configured to acquire monochromatic or polychromatic (e.g., based on a red-green-blue, or RGB, color model) images. Analysis of images acquired by such an optical imaging device 22 may detect facial expressions, or gestures or positions of other body parts (e.g., shoulders, arms, hands, or other body parts) that may be indicative of an emotional state of subject 14. Analysis of images acquired by such an optical imaging device 22 may detect changes in skin coloration that may be indicative of an emotional state of subject 14. In some cases, analysis of images acquired by an optical imaging device 22 may indicate eye movements or eyelid activity (e.g., indicative of alertness, drowsiness, or another state). In some cases, analysis of images acquired by an optical imaging device 22 may indicate relatively subtle skin surface movements that may be indicative of breathing rate, heartbeat or pulse, or other internal physiological functions.

An optical imaging device 22 may include a camera, optical sensor, or imaging device that is configured to acquired images in the infrared or another nonvisible spectral range. For example, images that are acquired by an optical imaging device 22 that acquires images in the thermal infrared spectral range be analyzed to yield a skin temperature of exposed skin (e.g., facial skin) of subject 14.

Subject state sensors 12 may include one or more physiological measurement sensors 23. Physiological measurement sensors 23 may include one or more sensors that are configured to measure one or more physiological functions or characteristics of subject 14.

Some types of physiological measurement sensor 23 may require attachment to the body of subject 14. For example, a physiological measurement sensor 23 in the form of a heartbeat or pulse sensor may be worn on a band that is placed around the wrist, leg, neck, or chest of subject 14, or that is directly attached to the body of subject 14 (e.g., using suction, adhesive, or otherwise). A galvanic skin response (GSR) sensor may be configured to sense skin conductivity (e.g., indicative of perspiration). Similarly, a physiological measurement sensor 23 to measure another physiological function (e.g., blood pressure, body temperature, breathing rate or pattern, muscle tension or movement, brain wave patterns, or another physiological function) may be placed on or near the body of subject 14.

A physiological measurement sensor 23 may be configured to communicate wirelessly with system controller 18, e.g., to avoid wires that may interfere with free movement of subject 14. In some cases, a physiological measurement sensor 23 may be incorporated into an object with which subject 14 is in contact. For example, a physiological measurement sensor 23 that does not require direct contact with the skin or body of subject 14 may be incorporated into an object that may be in close contact with subject 14. Such a measurement may include an acoustic heartbeat or pulse sensor, a breathing sensor, motion sensor, or other sensor configured to sense a sound wave or other wave or pulse that may be created by a physiological function being measured. Such a measurement may also include a body temperature, perspiration rate, or other physiological function that may affect (e.g., by conduction, diffusion, or otherwise) a sensor that is not in direct contact with the skin of subject 14. Suitable objects in which such a physiological measurement sensor 23 may be incorporated may include a seat back, armrest, headrest, seat, or other object that may be in contact that is sufficiently proximate (e.g., via clothing of subject 14) to subject 14 so as to enable measurement of the physiological function.

A sensor that requires contact with the skin of subject 14 (e.g., temperature sensor, GSR sensor, or other sensor) may be incorporated into an object that subject 14 may be expected to handle or touch during expected activities (e.g., steering wheel of a vehicle, handle of a machine, or other object). In this case, the incorporated physiological measurement sensor 23 may be in wired contact with system controller 18.

In some cases, subject state sensors 12 may include one or more sensors that sense actions or behavior of subject 14. For example, sensor communication module 36 may communicate with a monitoring system of a vehicle (e.g., that monitors such actions as speed, steering, deviations from lanes, proximity of other vehicles, or other driver behavior). In some cases, sensor communication module 36 may receive sounds that are sensed by a microphone (e.g., via a mobile telephone or other voice recording device) that include sounds or speech by subject 14.

In some cases, emotional state system 10 may include one or more environmental sensors 34. Environmental sensors 34 may be configured to monitor a current environment in which subject 14 is located. For example, environmental sensors 34 may include one or more temperature sensors (e.g., to measure an air temperature near subject 14), one or more sensors to measure an illumination level, one or more sensors to detect and measure a loudness of sounds or ambient noise, one or more humidity sensors, one or more sensors for detecting or measuring substances that are present in the surrounding atmosphere (e.g., pollutants, odors, or other airborne substances), sensors to measure air currents, or other sensors for measuring an environmental factor.

For example, system controller 18 may be configured to utilize measurements by environmental sensors 34 to adjust a calculation of an emotional state metric. For example, a sensed body temperature, heartrate, respiration rate, perspiration level, or other physiological parameter measured by one or more subject state sensors 12 may be adjusted in accordance with an ambient temperature, humidity level, or other environmental factor measured by environmental sensors 34. As another example, system controller 18 may monitor environmental changes due to operation of modification stimulation devices 16 and adjust operation of modification stimulation devices 16 in accordance with the monitored changes.

System controller 18 may include a sensor communication module 36 to operate subject state sensors 12 and to receive data from subject state sensors 12. Sensor communication module 36 may be configured to communicate with subject state sensors 12 via an electrical or optical cable, or wirelessly. For example, sensor communication module 36 may include one or more interfaces that are configured to send a control signal to a subject state sensor 12 or environmental sensor 34 (e.g., where operation of that subject state sensor 12 or environmental sensor 34 requires activation, e.g., as opposed to a subject state sensor 12 or environmental sensor 34 that operates continuously or passively), and to receive signals that are indicative of a measurement by each subject state sensor 12 or environmental sensor 34.

In some cases, sensor communication module 36 may include circuitry (e.g., an amplifier circuit, logic circuit, or other circuit) for enabling at least initial adjustment, calibration, or other interpretation or analysis of a received sensor signal. In some cases, at least some adjustments, calibrations, or analysis of sensor signals is performed by processing module 38.

Processing module 38 of system controller 18 may include processor 42 and data storage 38.

For example, processor 42 may include one or more processing units, e.g. of one or more computers. Processor 42 may be configured to operate in accordance with programmed instructions stored in data storage 44.

Data storage 44 may include one or more fixed or removable, volatile or nonvolatile, local or remote (e.g., at a remote server or cloud storage), memory or data storage devices. For example, data storage 44 may be utilized to store programmed instructions for operation of processor 42. Data storage 44 may be utilized to store data or parameters for use by processor 42 during operation, or results of operation of processor 42.

In particular, data storage 44 may be utilized to store one or more algorithms or formulae for calculating one or more emotional state metrics based on received signals from one or more subject state sensors 12 or environmental sensors 34. Data storage 44 may be utilized for storing one or more image analysis algorithms or one or more image features that may be used to identify a mood or emotional state from images acquired by an optical imaging device 22. Data storage 44 may be utilized to store parameters to identify one or more target emotional states.

An algorithm stored in data storage 44 may be based on psychological theory and empirical data to interpret raw data from one or more subject state sensors 12 as one or more emotions. In particular, an algorithm may be based on a valence-arousal model (for example, where high arousal and negative valence may be interpreted as indicating stress). An algorithm may calculate a stress level using a formula that considers muscle tension and eye aperture. For example, mild stress may be associated with greater muscle tension and wider eye aperture than relaxation. Higher levels of stress may be characterized by greater variability. For example, when in high stress, sensor readings may exhibit greater than average variance.

System controller 18 may include modification device control module 40. For example, modification device control module 40 may include one or more interfaces or drivers for enabling system controller 18 to control operation of one or more modification stimulation devices 16. Modification device control module 40 may be configured to communicate with each modification stimulation device 16 via an electrical or optical cable, or wirelessly. For example, when processing module 38 determines that one or more modification stimulation devices 16 are to be operated, processing module 38 may control operation of modification device control module 40 to control those modification stimulation devices 16.

Modification stimulation devices 16 may include one or more devices that are configured to generate a stimulus that may affect an emotional state of subject 14. For example, a stimulus that is generated by a modification stimulation device 16 may be visual, audible, olfactory, tactile or haptic, thermal, or otherwise.

For example, modification stimulation devices 16 may include one or more light sources 24. For example, a light source 24 may include one or more light-producing devices that may illuminate a region that is visible by subject 14. Such light-producing devices may include incandescent bulbs, fluorescent bulbs, light emitting diodes (LED), halogen lamps, gas-filled lamps, lasers (e.g., diode lasers), black body emitters, or other sources of light. A light-producing device may have a controllable brightness. A light-producing device, or a collection of light-producing devices, may have an adjustable color. For example, a color of an incandescent source may depend on a filament temperature. A color of a white light emitting device may be adjustable by use of a set of filters or spectral selection using a grating or prism. A color of light emitted by a collection of differently colored LED or laser sources may be adjusted by selective operation of the sources. The light that is emitted by light source 24 may be continuous, pulsed, strobed, or otherwise modulated.

Characteristics of light that is emitted by light source 24 may be selected in accordance with known effects of different types of illumination. For example, various colors have been associated with wakefulness or alertness, excitement, calmness, or otherwise affecting an emotional state. Similarly, various types of light modulations may have been demonstrated as variously affecting an emotional state.

In some cases, a light source 24 may include a pre-existing (e.g., prior to installation of emotional state system 10) lighting source, e.g., of a passenger compartment or driver cabin, of a room in which subject 14 is present, a dashboard, or other pre-existing lighting.

In some cases, modification stimulation devices 16 may include one or more audio generators 26. For example, audio generators 26 may include one or more sound playback devices (e.g., configured to replay one or more sounds that were previously recorded in one or more digital or analog formats), speakers, earphones, sound generators (e.g., music or sound synthesizers, buzzers, bells, sirens, horns, whistles, chimes, or other sound generators), radio receivers, telephones, or other devices that are configured to, alone or in combination, produce an audible sound.

For example, processing module 38 may determine that one or more audible stimuli are to be generated to change an emotional state of subject 14. For example, if a change in the emotional state toward a target emotional state requires a calming, an audio generator 26 may be operated via modification device control module 40 to produce a sound that has been demonstrated to have a calming effect (at least on a previously tested population), e.g., slow or quiet music or singing, nature sounds, or other sounds. If stimulation is required (e.g., to counter inattention or drowsiness), audio generators 26 may be operated to produce a sound that has been demonstrated to induce alertness or excitement (e.g., fast-paced music, an alarm signal, loud or commanding human speech, or another sound). Specific examples of audible stimuli that may be produced by audio generator 26 may include vocal intervention (e.g., a dialog with subject 14, e.g., based on principles of cognitive behavioral therapy), verbal games (e.g., trivia, classification, or other verbal games e.g., to increase arousal, reduce stress, or increase attention), music for relaxation, music for arousal (e.g., applying the “Mozart effect”), rhythmic music (e.g., to accompany breathing and clenching exercises), or other audible stimuli.

In some cases, an audio generator 26 may include one or more pre-existing (e.g., prior to installation of emotional state system 10 or installed independently of emotional state system 10) audio components. For example, an audio generator 26 may include a component of a vehicle audio system, an audio system or audio-visual system of a home, office, or plant, a computer or portable telephone, or another pre-existing audio component.

In some cases, modification stimulation devices 16 may include one or more olfactory stimulation devices 28. For example, an olfactory stimulation device 28 may include an atomizer, a canister or compartment that encloses a source of a scent and with controllable openings that may be opened or closed, or another device configured to release a scent into an ambient atmosphere. In some cases, olfactory stimulation device 28 may be configured to release a scent into a conduit or duct, or otherwise into an airflow that is generated by a ventilation system, climate control system, air conditioning system, or other system capable or generating an airflow around subject 14 (e.g., in a vehicle compartment, room, interior space, or otherwise).

For example, processing module 38 may determine that one or more olfactory stimuli are to be generated to change an emotional state of subject 14. For example, a change in the emotional state toward a target emotional state may be facilitated by one or more scents that may be released by operation of olfactory stimulation device 28 via modification device control module 40. Examples of scents that may be effective may include scents that are extracted from various biological sources or natural sources, or may be synthesized using one or more chemical processes. For example, a floral, fruity, or other scent that is generally considered to be pleasant may facilitate reduction of stress in subject 14. On the other hand, an acrid or otherwise unpleasant scent may induce alertness. Release of a scent may be controlled so as to avoid habituation to the scent by subject 14.

For example, an olfactory stimulation device 28 may be configured to diffuse a chemo-signal (e.g., to reduce aggression), or to use a fragrance to increase arousal, to serve as a positive reinforcement, or to reduce stress.

Modification stimulation devices 16 may include one or more haptic devices 30. A haptic device 30 may be configured to generate a mechanical vibration or motion that may be perceived by subject 14 as a tactile or haptic sensation. For example, haptic device 30 may be configured to vibrate at a controllable frequency or with controllable amplitude. A haptic device 30 may be configured, e.g., with one or more movable, or extendible and retractable, projections that may be extended to contact or poke the body of subject 14.

For example, a haptic device 30 may be incorporated into a back, seat cushion, armrest, headrest, desktop, tabletop, or other part of a seat or piece of furniture on which subject 14 is sitting, leaning, or is otherwise in physical contact. A haptic device 30 may be incorporated into a floor upon which subject 14 is standing, or upon which feet of subject 14 are resting. A haptic device 30 may be incorporated into an object (e.g., steering wheel, control handle, or other objet) that subject 14 is grasping. In some cases, a haptic device 30 may be incorporated into a belt, garment, hat, helmet, strap, or other object that may be worn by subject 14, e.g., when interacting with emotional state system 10.

For example, processing module 38 may determine that one or more haptic stimuli are to be generated to change an emotional state of subject 14. For example, if a change in the emotional state toward a target emotional state requires relaxing, haptic device 30 may be operated via modification device control module 40 to produce a soothing or relaxing vibration or massaging motion. If stimulation is required (e.g., to counter inattention or drowsiness), haptic device 30 may be operated to produce a poking, tickling, or other sensation that may be perceived as unpleasant or that may be otherwise invigorating.

For example, haptic stimuli may be generated to explicitly send conscious messages (e.g., vibrations or electric stimuli that may be expected to consciously prod or command the attention of subject 14), to implicitly or unconsciously affect a mood or behavior (e.g., without interrupting the behavior of subject 14), to reinforce or reward behavior (e.g., generating a pleasant massage), to enhance arousal (e.g., change a seat position or generate a massaging effect), to reduce stress (e.g., generate a massaging effect), or otherwise modify an emotional state.

Modification stimulation may include an adjustment of vehicle behavior, for example in an autonomous or semi-autonomous vehicle, such as vehicle speed, lane keeping assistance, in accordance with a current emotional state of the driver or passenger. For example, if passenger is suffering from motion sickness, the vehicle may be automatically controlled to decelerate or to avoid sharp maneuvers.

Modification stimulation devices 16 may include one or more atmosphere modification devices 32. For example, an atmosphere modification device 32 may be configured to effect a change in temperature, e.g., in an ambient atmosphere that is surrounding subject 14, or more directly to the body of subject 14. As another example, atmosphere modification device 32 may modify air circulation, humidify or dehumidify an ambient atmosphere around subject 14, or otherwise modify the atmosphere that surrounds subject 14.

For example, atmosphere modification device 32 may include a temperature control device, such as an air conditioner or climate control system of a vehicle cabin or compartment, or of a room or other interior space, within which subject 14 is located. The temperature control device may be configured to be controllable by system controller 18. Alternatively or in addition, atmosphere modification device 32 may include a localized temperature control device for heating or cooling subject 14. For example, atmosphere modification device 32 may include heating devices (e.g., electrical resistors or heating elements) or cooling devices (e.g., refrigeration coils, tubes tor circulation of cool or cooled liquid, or other localized cooling devices) that are incorporated into an object with which subject 14 is in contact or is near to. Such objects may include parts of a seat or other piece of furniture (e.g., seat cushion, back, armrest, headrest, desktop, or other part of a seat or piece of furniture), parts of a helmet, vest, or other object or article of clothing that may be worn by subject 14, a nearby part of an enclosure within which subject 14 is located (e.g., wall, floor, or ceiling, or other part of an enclosure), or another object.

For example, processing module 38 may determine that one or more thermal stimuli are to be generated to change an emotional state of subject 14. In some cases, a thermal stimulus may be selected on the basis of a currently measured ambient temperature, relative humidity, or other environmental feature that may be measured by one or more environmental sensors 34. For example, when environmental sensors 34 indicate that the current ambient atmosphere is warmer than a comfortable temperature range or that relative humidity is above a comfortable level, operation of atmosphere modification device 32 to cool subject 14 may induce relaxation or increase alertness in subject 14. On the other hand, when environmental sensors 34 indicate that the current ambient atmosphere is colder than a comfortable temperature range, operation of atmosphere modification device 32 to warm subject 14 may induce relaxation or increase alertness in subject 14.

Processing module 38 may be configured (e.g., programmed) with machine learning capability. For example, processing module 38 may be configured to receive measured data that are indicative of an emotional state of subject 14 from subject state sensors 12 via sensor communication module 36 concurrently with operation of one or more modification stimulation devices 16. Processing module 38 may be configured to analyze the received data and correlate any changes with operation of modification stimulation devices 16. The measured emotional state of subject 14 may be compared with a target emotional state. Processing module 38 may be configured to modify programmed instructions or parameters stored in data storage 44 in accordance with measured results of application of particular stimuli. For example, if operation of one or more of modification stimulation devices 16 is found to not change the emotional state toward a target emotional state, processing module 38 may be configured to alter operation of modification stimulation devices 16 from an originally programmed operation. By recording which type of operation of modification stimulation devices 16 achieves a desired result, e.g., modifying the emotional state toward the target state, and analyzing the results for a particular subject 14, emotional state system 10 may automatically self-adapt for effective operation for that particular subject 14.

In some cases, system controller 18 may be configured to communicate with one or more external facilities, servers, or computers via communications channel 46. For example, communications channel 46 may include a wired or wireless connection to one or more communications networks. For example, communications channel 46 may be configured to send data, e.g., that is accumulated by subject state sensors 12 during operation of modification stimulation devices 16, to one or more central facilities. Such a central facility may be configured to receive and analyze data from a plurality of emotional state systems 10. The central facility may be configured to utilize the received data to increase the accuracy of programmed instructions for operation of each processing module 38, for improving or expanding a database that is stored in data storage 44, or otherwise improve operation of emotional state system 10. In some cases, improvements may be communicated via communications channel 46 to individual emotional state system 10. In some cases, e.g., where part or all of data storage 44 includes remote storage, each system controller 18 may be configured to load programmed instructions via system controller 18 prior when starting to operate (e.g., after power is turned on).

In some cases, communications channel 46 may be utilized to report to a user of emotional state system 10. For example, results of monitoring the current emotional state of a subject 14 may be communicated to subject 14 or to a person who supervises or is otherwise responsible for the performance of subject 14.

Power for operating one or more components of emotional state system 10 may be provided by a line voltage, may be provided via a system (e.g., vehicle or machine) with which emotional state system 10 is associated, or may be provided by an internal power source (e.g., storage battery, generator, or other power source), that is incorporated into one or more components (e.g., system controller 18, modification stimulation devices 16, subject state sensors 12, or other components) of emotional state system 10.

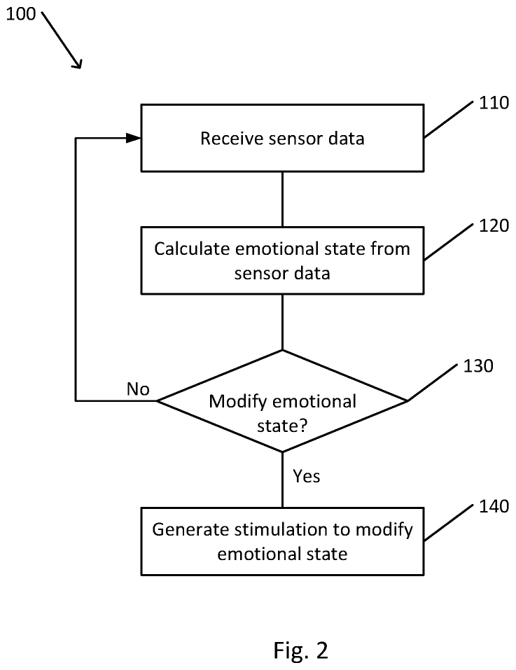

FIG. 2 is a flowchart depicting a method of operation of system for emotional state detection and modification, in accordance with an embodiment of the present invention.

It should be understood with respect to any flowchart referenced herein that the division of the illustrated method into discrete operations represented by blocks of the flowchart has been selected for convenience and clarity only. Alternative division of the illustrated method into discrete operations is possible with equivalent results. Such alternative division of the illustrated method into discrete operations should be understood as representing other embodiments of the illustrated method.

Similarly, it should be understood that, unless indicated otherwise, the illustrated order of execution of the operations represented by blocks of any flowchart referenced herein has been selected for convenience and clarity only. Operations of the illustrated method may be executed in an alternative order, or concurrently, with equivalent results. Such reordering of operations of the illustrated method should be understood as representing other embodiments of the illustrated method.

Emotional state modification method 100 may be executed by system controller 18 of emotional state system 10. For example, emotional state modification method 100 may be executed continuously when emotional state system 10, after the identity of a subject 14 has been entered or logged in, when a vehicle in which emotional state system 10 has been installed is operating, if a machine in a facility in which emotional state system 10 has been installed is operating, or under other circumstances. In some cases, prior to execution of emotional state modification method 100, system controller 18 may be configured to identify subject 14. For example, in some cases, subject 14 may be requested to enter identifying data prior to execution of emotional state modification method 100. Identification data may be entered by entering an alphanumeric identification (e.g., user name, password, or code), holding a personal identification card, badge, or other object (e.g., containing identification data encoded in an optical barcode or other optically detectable pattern or design, on a radiofrequency identification circuit, on a magnet strip, on an integrated circuit chip, or otherwise) to an appropriate reader, enabling a scanner to biometrically scan a body part (e.g., fingerprint, retina, or other) of subject 14, or may be otherwise entered. In some cases, system controller 18 may be configured to analyze data from one or more subject state sensors 12 (e.g., from one or more optical imaging devices 22) to automatically identify subject 14 (e.g., by applying face recognition technology or otherwise).

Sensor data may be received from one or more subject state sensors 12 by processing module 38 of system controller 18, e.g., via sensor communication module 36 (block 110). The sensor data may include image data from one or more optical imaging devices 22 that are configured to image at least a face of subject 14. In addition, the sensor data may include data from one or more physiological measurement sensors 23. Typically, at least one physiological measurement sensor 23 may be configured to measure heartbeat or pulse. One or more other physiological measurement sensors 23 may be configured to measure one or more other physiological parameters of subject 14. The sensed imaged or physiological parameters may be biometric features that are, at least in some cases, correlated with or affected by changes in an emotional state of subject 14.

In addition, in some cases, sensor data may be received from one or more environmental sensors 34.

The received sensor data may be stored in data storage 44 of processing module 38.

Processor 42 of processing module 38 may operate in accordance with programmed instructions, e.g., as stored on data storage 44, to calculate an emotional state from the received sensor data (block 120).

For example, one or more algorithms known in the art for determining an emotional state may be applied to image data (e.g., of the face of subject 14), heartbeat data, or other physiological data to calculate one or more metrics that are indicative of an emotional state. For example, in some cases, the calculation may yield a calculated emotional valence, a metric indicative of arousal, or a metric indicative of one or more other components of an emotional state.

The calculated current emotional state of subject 14 may be compared with a target emotional state to determine if the current emotional state is to be modified (block 130). For example, characteristics or metrics that are indicative of a target emotional state may be stored in data storage 44. A target emotional state may be determined on the basis of a range of emotional state metrics that are predetermined as suitable for subject 14. For example, the range of emotional state metrics may be determined to be suitable for a subject 14 who is performing a particular task, such as driving a vehicle or operating machinery. In some cases, the range of emotional state metrics may be predetermined as suitable for a subject 14 (e.g., performing a particular task or otherwise) under one or more sensed environmental conditions (e.g., noise level, atmospheric conditions, lighting conditions, or other environmental conditions).

An algorithm may be applied to determine if modification is indicated. An example of an algorithm may be written as the following pseudocode formula:

if[average(Surprise)>norm(Surprise) and average(HR)>norm(HR)+normSD(HR)] or [average(Engagement)>norm(Engagement) and average(HR)>norm(HR)+normSD(HR)]then trigger-intervention(Stress)

In this formula, average indicates an average over a predetermined period of time prior to the current time (e.g., X seconds), SD indicates the standard deviation over the predetermined period of time, norm indicates an average value during a baseline period (e.g., where subject 14 is relaxed), normSD indicates a standard deviation during the baseline period, Surprise is an emotional state metric indicative of surprise, Engagement is an emotional state metric indicative of engagement, HR indicates heart rate, trigger-intervention indicates that a modification is indicated, and Stress indicates that the modification is for excessive stress.

Another example of an algorithm:

if[average(Surprise)>1.5*norm(Surprise) and average(HR)>norm(HR)+normSD(HR)]then trigger-intervention(Mild-Stress)

where Mild-Stress indicates that the modification is for mild stress.

If no modification of the emotional state is indicated, sensor data may continue to be received in order to monitor the emotional state of subject 14 and to continue to compare the monitored emotional state with a target emotional state (block 110).

If modification of the emotional state is indicated, system controller 18 may operate one or more modification stimulation devices 16 via modification device control module 40 to generate one or more stimuli (block 140). For example, a stimulus may be selected by processing module 38, e.g., in accordance with a database stored on data storage 44 of processing module 38. The database may correlate stimuli with previously measured changes in emotional state. Typically, the correlations between stimuli and changes in emotional state may be compiled based on previous measurements on a large, e.g., statistically significant, and typically diverse (e.g., of differing ages, gender, ethnic or cultural background, socioeconomic status, family status, body type or build, or other sources of diversity) population of subjects.

For example, when stress is indicated, modification may include verbal instructions, or other audible cues, to instruct the subject to breath in a manner that is likely to reduce stress (e.g., inhale for 3 seconds, hold breath for 3 seconds, and exhale slowly for 6 seconds). In some cases, a modification may be prefaced by one or more inquiries (e.g., a structured series of questions and responses) to determine whether modification is in fact required.

In some cases, a stimulus may be generated in accordance with a predicted emotional state. For example, current sensor readings and calculated metrics, which are indicative of a current emotional state, may also be predictive of a predetermined undesirable future emotional state that is to be avoided. System controller 18 may be configured to predict the undesirable future emotional state on the basis of the current sensor readings, calculated metrics, or both. System controller 18 may be configured to operate modification stimulation devices 16 to generate a stimulus that has been previously identified as preventing or inhibiting the change for the current emotional state to the undesirable predicted emotional state. For example, a particular sensor reading may be indicative of a current relaxed state but may indicate an increase in emotional stress in a few minutes. System controller 18 may be configured to generate a stimulus for the purpose of preventing or easing the predicted increase in emotional stress.

Typically, as the stimuli are being generated by modification stimulation devices 16, system controller 18 may continue to receive data from subject state sensors 12 so as to continue to monitor the current emotional state of subject 14 as the state is modified by the stimuli. In some cases, the monitoring may indicate that the modified current emotional state has reached, e.g., is identical or similar to, the target emotional state. For example, the current emotional state may be considered to have reached the target emotional state when one of more metric values that are calculated from the sensor data are within a predetermined range of the values that characterize the target emotional state.

In some cases, when the monitored emotional state of subject 14 reaches the target emotional state, generation of the stimulus by modification stimulation devices 16 may be stopped. In some cases, generation of a stimulus by modification stimulation devices 16 may continue after the modified current emotional state has reached the target emotional state. For example, system controller 18 may be configured to continue operation of one or more modification stimulation devices 16 to generate stimuli when the generated stimuli have been previously determined to maintain the current emotional state at the target emotional state. On the other hand, system controller 18 may be configured to stop operation of one or more modification stimulation devices 16 when continued generation of the associated stimuli has been previously determined to overcorrect or otherwise change the current emotional state from the target emotional state (e.g., a stimulus may become annoying rather than continuing to be calming or energizing, or may otherwise become evocative of other emotions). Other considerations as to whether or not to continue operation of a modification stimulation device 16 may include a likelihood that subject 14 may become habituated to continued application of the stimulus, a likelihood that continued generation of the stimulus may become disturbing or disruptive to other people in the vicinity of subject 14 (e.g., passengers in a vehicle that is being driven by subject 14, coworkers in the vicinity of subject 14, or other bystanders), or may otherwise result in an undesirable effect.

In some cases, processing module 38 may be configured to apply machine learning to data that is received from subject state sensors 12 during operation of modification stimulation devices 16. For example, processing module 38 may be configured to analyze the effect of one or more stimuli generated by modification stimulation devices 16 on a particular subject 14. The analysis may determine whether or not one or more of the stimuli are effective in modifying the current emotional state of subject 14 in an expected manner (e.g., based on a general population sample). In the event that the effect of the stimulus is not as expected, system controller 18 may, in future executions of emotional state modification method 100, modify the stimuli that are applied to subject 14 and monitor the results. In this manner, system controller 18 may be configured to self-adapt the operation of modification stimulation devices 16 to a particular subject 14 (or to users of a particular emotional state system 10, e.g., when emotional state system 10 is not configured to identify individual subjects 14).

In some cases, processing module 38 may be further configured to communicate results of one or more executions of emotional state modification method 100 to an appropriate remote device or system. For example, reporting on a monitored emotional state of a subject 14 who is driving a vehicle may assist in helping that subject 14 to modify any potentially unsafe behaviors or habits. Collected data regarding emotional states and behaviors of subjects 14 be reported (e.g., to a publicly accessible communications channel or location) so as to benefit of other users (e.g., drivers), equipment manufacturers, businesses, and licensing, safety inspection, law enforcement, legislators, or other authorities.

In some cases, an emotional state system 10 may be configured for operation with an autonomous vehicle operation system.

For example, such an autonomous vehicle operation system could enable a driver to engage in various activities such as reading, watching a movie, playing games, or other activities while the vehicle continues to travel along toward a predetermined destination. Engagement is such activities may increase the likelihood that the driver may suffer from motion sickness. When indications of motion sickness, or indications of conditions that may lead to motion sickness, are detected by subject state sensors 12, modification stimulation devices 16 may be operated to decrease the likelihood or severity of motion-sickness. For example, modification stimulation devices 16 may be operated to generate visual cues that bring perceived motion into conformity with actual vehicle motion, thus possibly decreasing the likelihood of motion sickness. As another example, a fragrance that is emitted by olfactory stimulation device 28 may ease symptoms of motion sickness.

As another example, emotional state system 10 may detect with a driver of the autonomous vehicle (or another, e.g., driver operated or otherwise non-autonomous vehicle) is drowsy or asleep. In such a case, emotional state system 10 may be configured such that if emotional state system 10 cannot awaken the driver, emotional state system 10 may operate an exterior alert system (e.g., that generates externally visible lights and externally audible sounds) to alert other drivers that the driver is asleep.

Different embodiments are disclosed herein. Features of certain embodiments may be combined with features of other embodiments; thus, certain embodiments may be combinations of features of multiple embodiments. The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A system for monitoring and modifying an emotional state of a subject, the system comprising:
at least one sensor configured to sense data indicative of a plurality biometric features of a subject that are related to a current emotional state of the subject;
at least one stimulation device that is configured to generate a stimulus to modify the current emotional state; and
a processor configured to receive the sensed data, to use the sensed data to calculate at least one metric that is indicative of the current emotional state of the subject, to identify a value of said at least one metric that is indicative of a predetermined target emotional state, and, when the indicated current emotional state is different from the target emotional state, to operate said at least one stimulation device to generate a stimulus that has been previously identified as facilitating changing the current emotional state to the target emotional state.

2. The system of claim 1, wherein said at least one sensor comprises an optical imaging device, and the sensed data comprises an image.

3. The system of claim 2, wherein the biometric feature comprises a facial expression, and wherein the processor is further configured to analyze one or more images acquired by the optical imaging device to detect the facial expression and to calculate said at least one metric on the basis of the detected facial expression.

4. The system of claim 3, wherein said at least one sensor comprises a physiological sensor configured to sense at least one physiological parameter of the subject.

5. The system of claim 4, wherein the physiological sensor comprises a heartbeat sensor, and the physiological function comprises a heartbeat or pulse of the subject.

6. The system of claim 4, wherein the physiological sensor comprises a galvanic skin response sensor, and the physiological function comprises a conductivity of skin of the subject.

7. The system of claim 1, wherein said at least one stimulation device comprises a light source.

8. The system of claim 7, wherein the processor is configured to control brightness or color of light that is emitted by the light source.

9. The system of claim 1, wherein said at least one stimulation device comprises an olfactory stimulation device to emit an olfactory stimulus.

10. The system of claim 1, wherein said at least one stimulation device comprises an audio generator to generate an audible stimulus.

11. The system of claim 1, wherein said at least one stimulation device comprises a haptic device to generate a tactile stimulus.

12. The system of claim 1, wherein said at least one stimulation device comprises a temperature control device.

13. The system of claim 1, wherein the system is installable in a vehicle, wherein the subject is a driver of the vehicle.

14. The system of claim 1, wherein the processor is further configured to use the sensed data to predict a future emotional state of the subject.

15. The system of claim 14, wherein when the predicted indicated emotional state is a predetermined undesirable emotional state, and wherein the processor is further configured to operate said at least one stimulation device to generate a stimulus that has been previously identified as inhibiting a change of the current emotional state to the undesirable emotional state.

16. A method for monitoring and modifying an emotional state of a subject, the method comprising, by a processor:
receiving sensed data from at least one sensor, the sensed data indicative of a plurality of biometric features of a subject that are related to a current emotional state of the subject;
calculating, using the sensed data, at least one metric that is indicative of the current emotional state;
identifying a value of said at least one metric that is indicative of a predetermined target emotional state; and
when the indicated current emotional state is different from the target emotional state, operating at least one stimulation device to generate a stimulus that has been previously identified as facilitating changing the current emotional state to the target emotional state.

17. The method of claim 16, wherein a biometric feature of the plurality of biometric features comprises a facial expression, and the sensed data comprises one or more images acquired by an optical imaging device.

18. The method of claim 16, wherein a biometric feature of the plurality of biometric features comprises a heartbeat or skin conductivity.

19. The method of claim 16, wherein the at least one stimulation device is selected from a group of stimulation device consisting of: a light source, an olfactory stimulation device, audio generator, a haptic device and a temperature control device.

20. The method of claim 16, further comprising predicting a future emotional state of the subject and, when the predicted indicated emotional state is a predetermined undesirable emotional state, operating said at least one stimulation device to generate a stimulus that has been previously identified as inhibiting a change of the current emotional state to the undesirable emotional state.
